(19) United States
(12) Patent Application Publication
(10) Pub. No.: US 2012/0035435 A1
(43) Pub. Date: Feb. 9, 2012

(54) ELECTROCARDIOGRAM MONITOR

(76) Inventors: Minkyung Grace Choi, Bayside, NY (US); Martin R. Prince, New York, NY (US)

(21) Appl. No.: 13/147,072

(22) PCT Filed: Jan. 28, 2010
(86) PCT No.: PCT/US2010/022313

§ 371 Date: Oct. 7, 2011

Related U.S. Application Data
(60) Provisional application No. 61/148,364, filed on Jan. 29, 2009.

(51) Int. Cl. A61B 5/0408; A61B 5/08

(52) U.S. Cl. 600/301; 600/393; 600/390; 600/382

(57) ABSTRACT

A device is provided for sensing an electrocardiogram signal of a patient. The device includes a substrate and a plurality of electrodes mounted to the substrate. The plurality of electrodes are configured to sense an electrocardiogram signal of a patient when the plurality of electrodes are placed in contact with the patient. The device may include a strap configured to wrap around a portion of the patient wherein the substrate mounts to the strap. The device further may include a respiration sensor mounted to the strap.

### CROSS-REFERENCE TO RELATED APPLICATIONS

The present application claims priority to U.S. Provisional Patent Application Ser. No. 61/148,364, filed on Jan. 29, 2009, and titled “COMBINED ELECTROCARDIOGRAM AND RESPIRATORY MONITOR,” the disclosure of which is incorporated herein by reference in its entirety.

### BACKGROUND

Electrocardiogram (ECG) gating is used in a wide variety of medical imaging procedures, including but not limited to computed tomography (CT), positron emission tomography (PET), ultrasound, and magnetic resonance imaging (MRI). ECG gating is used in MRI primarily for cardiac MRI and also for magnetic resonance angiography (MRA) and other magnetic resonance techniques where it is necessary to synchronize data acquisition with cardiac contraction. ECG and respiratory monitoring may also be used when a patient is having general anesthesia, sedation, or a contrast agent injection during MRI to monitor vital signs.

When cardiac MRI is performed, it is necessary for the magnetic resonance (MR) scanner to detect the ECG signal from the patient so that imaging can be synchronized to the heartbeat. Without an effective mechanism to detect an ECG signal, these techniques are degraded by heart motion and may not be diagnostic. Some alternatives to cardiac imaging without ECG gating involve very fast scanning, which is fast enough to freeze the motion of the heart. Another alternative is termed “self-gating” where the heart motion or tissue/artery pulsation is extracted from the image data. These alternative methods place limitations on the scanning, but are still used because currently the use of ECG gating is cumbersome, unreliable, and time consuming adding expense and inconvenience to the patient due to the time to properly position the electrodes/leads on the patient.

Currently, ECG gating is performed by placing three or four leads on the patient's chest. These leads (sometimes called electrodes) preferably are made out of non-ferrous conducting material to avoid metal artifact. The leads should be sufficiently large in diameter to avoid causing a burn from excessive conduction of current through too small an area of the skin. Typically, at least 2 centimeters (cm) in diameter is sufficient to avoid burns from excessive electrical resistance at the lead-skin interface.

Because the MRI scanner environment is electrically noisy with noise from gradient activity, RF activity, and muscle enervation related to patient movements, it can be very difficult to place the electrodes in the optimum position for receiving a sufficiently strong signal that can be detected distinctly from the superimposed electrical noise. As a result, after placing all four leads, it may be necessary to adjust the position to find positions that provide a stronger signal. Also, it is important that the electrodes have a very strong stick mechanism, typically a sticky glue, to keep the electrode attached to the skin on the chest. If the electrode becomes unattached, it will not function properly. Additionally, because electrical wires can heat up in the MRI scanner due to a developed oscillating current, the wires need to be carefully positioned so that they do not touch the skin, but are as straight as possible to avoid loops that can pick up current from the oscillating magnetic fields of the MRI scanner.

After placing the leads on the chest, the leads are joined to a cable that exits the bore of the scanner and is plugged into a port on the scanner. The process of connecting the leads can be somewhat confusing because the labeling of the leads (right arm, left arm, right leg, left leg) often does not match up well to the position of the four electrodes. Confusion in connecting the electrodes to the ECG cable may slow down the process of connecting the wires, delaying the scan, and reducing the number of patients per day that can be scanned using an imaging machine. This adds cost to the examinations which require ECG gating.

Whenever ECG gating is necessary, it is frequently also necessary to monitor the patient's breathing with a respiratory monitoring device such as a respiratory bellows. This helps with properly instructing the patient in breath holding during scanning by allowing the operator to observe whether the patient is following (or not following) breathing instructions. Respiratory monitoring also may be used for respiratory gating the MRI scan to reduce respiratory motion artifact.

Thus, after the ECG leads are connected, a device for monitoring respiration may be wrapped around the chest or abdomen. The device is usually a hollow tube that can stretch like an accordion. As its length changes with inspiration and expiration, the hollow tube drives air through a tube which can then be detected to monitor the breathing. However the standard bellows used with MRI scanners, typically a one cm circular tube, frequently does not have sufficient airflow to be sensitive enough to detect breathing in all patients. Accordingly, it is often necessary to readjust the respiratory bellows position or its strap in order to improve the signal. The respiratory bellows on the front of the chest can also interfere and knock loose the ECG leads which are placed on the front of the chest. Although the respiratory bellows are reusable from one patient to the next, the ECG electrodes are disposable, which adds cost.

### SUMMARY

In an example embodiment, a device is provided for sensing an electrocardiogram signal of a patient. The device includes, but is not limited to, a substrate and a plurality of electrodes mounted to the substrate. The plurality of electrodes are configured to sense an electrocardiogram signal of a patient when the plurality of electrodes are placed in contact with the patient. The device may include a strap configured to wrap around a portion of the patient wherein the substrate mounts to the strap. The device further may include a respiration sensor mounted to the strap.

In another example embodiment, a diagnostic system is provided. The diagnostic system includes, but is not limited to, an ECG sensor device, a scanner configured to generate data related to a physiological characteristic of a patient, and a computing device. The ECG sensor device includes, but is not limited to, a substrate and a plurality of electrodes mounted to the substrate. The plurality of electrodes are configured to sense an electrocardiogram signal of the patient when the plurality of electrodes are placed in contact with the patient. The computing device includes a communication interface, a processor operably coupled to the communication interface, and a computer readable medium operably coupled to the processor. The communication interface is configured to receive the sensed electrocardiogram signal from the ECG sensor device. The computer-readable medium has computer-readable instructions stored thereon that, when executed by the processor, cause the system to control operation of the scanner based on the received electrocardiogram signal.

The foregoing summary is illustrative only and is not intended to be in any way limiting. Other principal features and advantages of the invention will become apparent to those skilled in the art upon review of the following drawings, the detailed description, and the appended claims.

### BRIEF DESCRIPTION OF THE DRAWINGS

Example embodiments will hereafter be described with reference to the accompanying drawings, wherein like numerals denote like elements. The drawings depict example embodiments in accordance with the disclosure and are, therefore, not to be considered limiting of its scope.

FIG. 1 depicts a block diagram of an example embodiment of a patient diagnostic system.

FIG. 2 depicts an ECG sensor device of the patient diagnostic system of FIG. 1 in accordance with a first example embodiment.

FIG. 3 depicts an ECG sensor device of the patient diagnostic system of FIG. 1 in accordance with a second example embodiment.

FIG. 4 depicts an ECG sensor device of the patient diagnostic system of FIG. 1 in accordance with a third example embodiment.

FIG. 5 depicts an ECG sensor device of the patient diagnostic system of FIG. 1 in accordance with a fourth example embodiment.

FIG. 6 depicts an ECG sensor device of the patient diagnostic system of FIG. 1 in accordance with a fifth example embodiment.

FIG. 7 depicts a combined ECG and respiration sensor device of the patient diagnostic system of FIG. 1 in accordance with an example embodiment.

### DETAILED DESCRIPTION

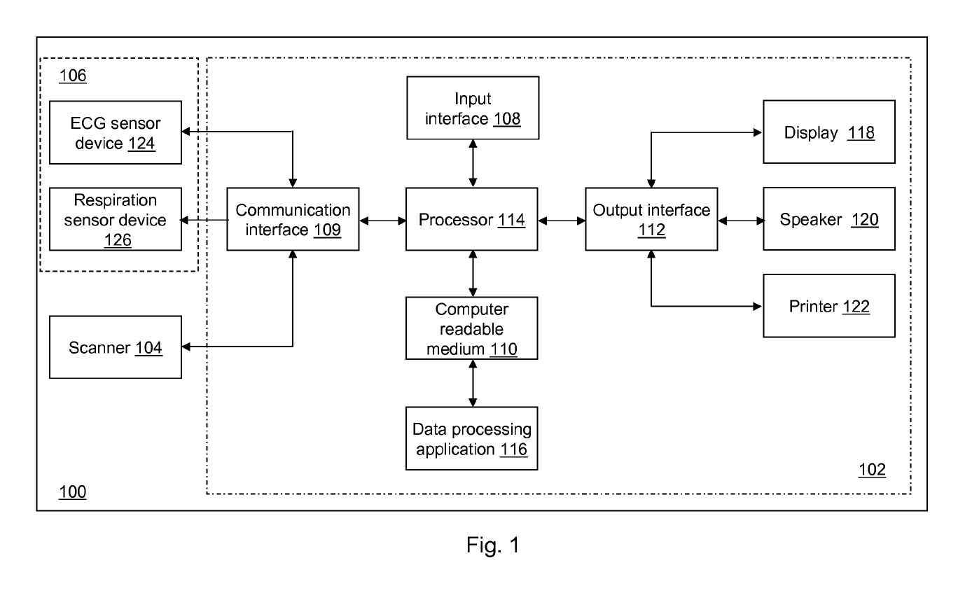

With reference to FIG. 1, a block diagram of a patient diagnostic system 100 is shown in accordance with an example embodiment. Patient diagnostic system 100 may include a computing system 102, a scanner 104, and an ECG/respiration sensor device 106. Different and additional components may be incorporated into patient diagnostic system 100. Computing system 102 may include an input interface 108, a communication interface 109, a computer-readable medium 110, an output interface 112, a processor 114, a data processing application 116, a display 118, a speaker 120, and a printer 122. Different and additional components may be incorporated into computing system 102.

Input interface 108 provides an interface for receiving information from the user for entry into computing system 102 as known to those skilled in the art. Input interface 108 may use various input technologies including, but not limited to, a keyboard, a pen and touch screen, a mouse, a track ball, a touch screen, a keypad, one or more buttons, etc. to allow the user to enter information into computing system 102 or to make selections presented in a user interface displayed on display 118. The same interface may support both input interface 108 and output interface 112. For example, a touch screen both allows user input and presents output to the user. Computing system 102 may have one or more input interfaces that use the same or a different input interface technology.

Communication interface 109 provides an interface for receiving and transmitting data between devices using various protocols, transmission technologies, and media as known to those skilled in the art. Communication interface 109 may support communication using various transmission media that may be wired or wireless. Computing system 102 may have one or more communication interfaces that use the same or a different communication interface technology. Data and messages may be transferred between computing system 102, scanner 104, and/or ECG/respiration sensor device 106 using communication interface 109.

Computer-readable medium 110 is an electronic holding place or storage for information so that the information can be accessed by processor 114 as known to those skilled in the art. Computer-readable medium 110 can include, but is not limited to, any type of random access memory (RAM), any type of read only memory (ROM), any type of flash memory, etc. such as magnetic storage devices (e.g., hard disk, floppy disk, magnetic strips, . . . ), optical disks (e.g., CD, DVD, . . . ), smart cards, flash memory devices, etc. Computing system 102 may have one or more computer-readable media that use the same or a different memory media technology. Computing system 102 also may have one or more drives that support the loading of a memory media such as a CD or DVD. Computer-readable medium 110 may provide the electronic storage medium for scanner 104 and/or ECG/respiration sensor device 106. Computer-readable medium 110 further may be accessible to computing system 102 through communication interface 109.

Output interface 112 provides an interface for outputting information for review by a user of computing system 102. For example, output interface 112 may include an interface to display 118, speaker 120, printer 122, etc. Display 118 may be a thin film transistor display, a light emitting diode display, a liquid crystal display, or any of a variety of different displays known to those skilled in the art. Speaker 120 may be any of a variety of speakers as known to those skilled in the art. Printer 122 may be any of a variety of printers as known to those skilled in the art. Computing system 102 may have one or more output interfaces that use the same or a different interface technology. Display 118, speaker 120, and/or printer 122 further may be accessible to computing system 102 through communication interface 109.

Processor 114 executes instructions as known to those skilled in the art. The instructions may be carried out by a special purpose computer, logic circuits, or hardware circuits. Thus, processor 114 may be implemented in hardware, firmware, or any combination of these methods and/or in combination with software. The term “execution” is the process of running an application or the carrying out of the operation called for by an instruction. The instructions may be written using one or more programming language, scripting language, assembly language, etc. Processor 114 executes an instruction, meaning that it performs/controls the operations called for by that instruction. Processor 114 operably couples with input interface 108, with communication interface 109, with computer-readable medium 110, and with output interface 112, to receive, to send, and to process information. Processor 114 may retrieve a set of instructions from a permanent memory device and copy the instructions in an executable form to a temporary memory device that is generally some form of RAM. Computing system 102 may include a plurality of processors that use the same or a different processing technology.

Data processing application 116 performs operations associated with processing data for a patient gathered using one or more electronic devices that continuously, periodically, and/or upon request monitor, sense, measure, etc. the physiological characteristics of a patient. In an example embodiment, the data is obtained from a medical imaging system such as an MRI device, a CT scanner, a PET scanner, an ultrasound machine, an X-ray machine, etc., from a sensor associated with measuring a physiological characteristic of a patient such as a temperature, a blood pressure, a heart rate, blood chemistry, a respiratory rate, a heart state or condition, an intra-abdominal pressure, etc., from medical personnel evaluating and treating the patient, etc. The operations may be implemented using hardware, firmware, software, or any combination of these methods. With reference to the example embodiment of FIG. 1, data processing application 116 is implemented in software (comprised of computer-readable and/or computer-executable instructions) stored in computer-readable medium 110 and accessible by processor 114 for execution of the instructions that embody the operations of data processing application 116. Data processing application 116 may be written using one or more programming languages, assembly languages, scripting languages, etc.

Scanner 104 may include a medical imaging system such as an MRI device, a CT scanner, a PET machine, an X-ray machine, an ultrasound device, etc. Scanner 104 generates data related to a patient in two-dimensions, three-dimensions, four-dimensions, etc. The source of and the dimensionality of the data is not intended to be limiting. Computing system 102 may be separate from or integrated with scanner 104 to control the operation of scanner 104.

ECG/respiration sensor device 106 may include an ECG sensor device 124 and a respiration sensor device 126. Different and additional components may be incorporated into ECG/respiration sensor device 106. ECG sensor device 124 detects/senses an ECG signal generated by the heartbeat of the patient. Respiration sensor device 126 detects/senses an inspiration/respiration of the patient.

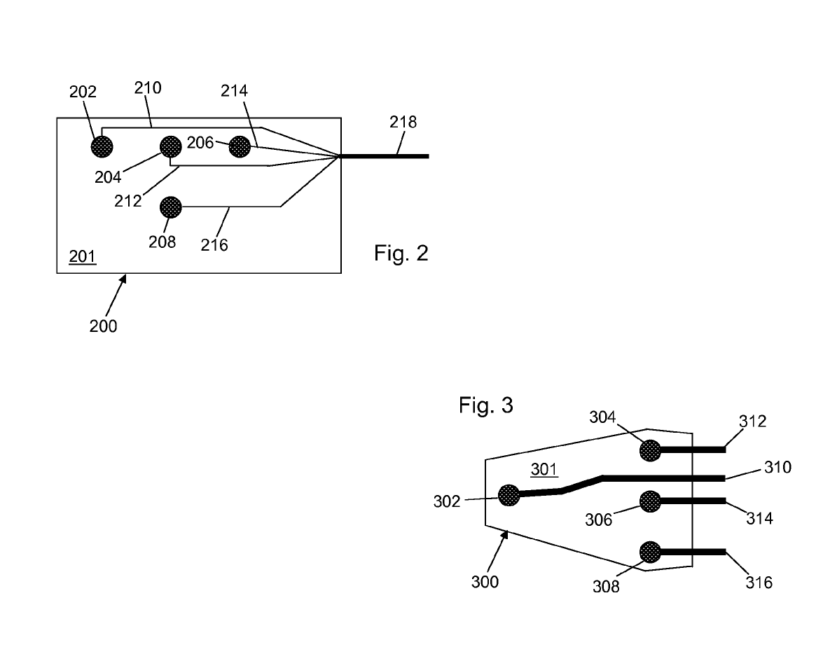

With reference to FIG. 2, an ECG sensor device 200 is shown in accordance with a first example embodiment. ECG sensor device 200 may include a substrate 201, a first electrode/lead 202, a second electrode/lead 204, a third electrode/lead 206, a fourth electrode/lead 208, a first wire 210, a second wire 212, a third wire 214, a fourth wire 216, and a cable 218. ECG sensor device 200 combines the ECG electrodes/leads on a single device for rapid application to the patient. ECG sensor device 200 may include a fewer or a greater number of electrodes and corresponding wires.

Substrate 201 may comprise a non-conductive material such as non-conductive silicone, silicon rubber, plastic, or other electrically insulating material. Substrate 201 further may comprise a biocompatible flexible material, which is deformable, such as a foam. In an example embodiment, a viscoelastic foam may be used. Substrate 201 may be encased in a film which prevents fluids from contacting, for example, the foam, and makes ECG sensor device 200 easy to clean between patients. In an example embodiment, a covering made of plastic film such as Dacron or CRYPTON fabric such as that manufactured by Crypton, Inc. is used. A heat shrink covering may also be applied to form substrate 201. Substrate 201 may be formed in a variety of shapes including circles, ellipses, polygons, etc. having a variety of sizes sufficient to accommodate the desired number and arrangement of electrodes/leads and corresponding wires.

First electrode/lead 202, second electrode/lead 204, third electrode/lead 206, and fourth electrode/lead 208 are mounted on a surface of substrate 201. As used herein, the term “mount” includes join, unite, connect, associate, insert, hang, hold, affix, attach, fasten, bind, paste, secure, bolt, screw, rivet, solder, weld, press against, formed with, glue, clip, layer, etch, and other like terms. For example, the material forming first electrode/lead 202, second electrode/lead 204, third electrode/lead 206, and fourth electrode/lead 208 may be incorporated into substrate 201 where it is desired to have an electrode with a conductive path inside substrate 201 similar to the manner in which conductive tracts are incorporated into silicon electronic chips or electrical circuit boards.

First electrode/lead 202, second electrode/lead 204, third electrode/lead 206, and fourth electrode/lead 208 may be formed of a conductive, non-metallic material. In example embodiments, first electrode/lead 202, second electrode/lead 204, third electrode/lead 206, and fourth electrode/lead 208 are formed of conductive silicon rubber, conductive epoxy, carbon black, carbon rubber, etc.

First electrode/lead 202, second electrode/lead 204, third electrode/lead 206, and fourth electrode/lead 208 are sufficiently separated from one another to prevent short circuits between them. Typically, at least one cm is a sufficient spacing between each of first electrode/lead 202, second electrode/lead 204, third electrode/lead 206, and fourth electrode/lead 208. First electrode/lead 202, second electrode/lead 204, third electrode/lead 206, and fourth electrode/lead 208 may be formed in a variety of shapes including circles, ellipses, polygons, etc.

First electrode/lead 202, second electrode/lead 204, third electrode/lead 206, and fourth electrode/lead 208 may be formed having a variety of sizes. Normally, ECG electrodes/leads have a small cross-sectional area to precisely define the point source of the signal. However, because MRI and other scanning devices do not require knowledge of the source of the ECG signal with high precision, large cross-sectional area electrodes/leads can be used to provide a better electrical contact with the skin possibly eliminating the need for use of conducting gel or for precise placement of the electrode/lead. Additionally, skin preparation is not necessary using a large cross-sectional area for the electrodes/leads, and the risk of burns is decreased. In the example embodiment of FIG. 2, first electrode/lead 202, second electrode/lead 204, third electrode/lead 206, and fourth electrode/lead 208 are generally circular and have a diameter of approximately two centimeters and preferably three or four centimeters though larger diameters may be used.

First electrode/lead 202, second electrode/lead 204, third electrode/lead 206, and fourth electrode/lead 208 may be arranged to form an array having a variety of shapes. In the example embodiment of FIG. 2, first electrode/lead 202, second electrode/lead 204, third electrode/lead 206, and fourth electrode/lead 208 are arranged to form a generally T-shaped array though such an arrangement is not intended to be limiting. First electrode/lead 202, second electrode/lead 204, third electrode/lead 206, and fourth electrode/lead 208 are arranged to provide a sufficient signal related to the heartbeat of the patient.

A deformable material may be positioned between each electrode 202, 204, 206, 208 and substrate 201 to ensure a good contact with the skin of the patient. In an example embodiment, the deformable material is a foam, which is one to ten cm thick. A viscoelastic foam may be particularly comfortable. A deeper foam, for example, five to ten cm thick or more may be used to allow first electrode/lead 202, second electrode/lead 204, third electrode/lead 206, and fourth electrode/lead 208 to make good contact with the skin.

The patient may lay on ECG sensor device 200 so that the skin contacts first electrode/lead 202, second electrode/lead 204, third electrode/lead 206, and fourth electrode/lead 208 to sense/measure an ECG signal of the heartbeat of the patient, for example, for ECG gating. Conductivity between the skin and first electrode/lead 202, second electrode/lead 204, third electrode/lead 206, and fourth electrode/lead 208 may be enhanced using a conductive gel. Use of a conductive gel may allow conduction through a gown such that removal of the gown may not be necessary. If the gown is removed, the bare skin can be prevented from directly touching ECG sensor device 200 by placing a thin paper or other material between the patient and ECG sensor device 200. The paper may be porous to the conductive gel placed on electrodes 202, 204, 206, 208 such that the conductive gel assists in the conduction of the electrical signal through the paper at the sites of electrodes 202, 204, 206, 208. ECG sensor device 200 is easily cleaned for reuse by removing and replacing the paper between patients. No adhesive and no shaving is required to make adequate contact between the electrode/lead and the skin of the patient.

First electrode/lead 202, second electrode/lead 204, third electrode/lead 206, and fourth electrode/lead 208 are connected or mount to first wire 210, second wire 212, third wire 214, and fourth wire 216, respectively, so that first wire 210, second wire 212, third wire 214, and fourth wire 216 conduct a signal detected by first electrode/lead 202, second electrode/lead 204, third electrode/lead 206, and fourth electrode/lead 208, respectively. The material forming first wire 210, second wire 212, third wire 214, and fourth wire 216 may be incorporated into substrate 201 where it is desired to define a conductive path inside substrate 201 similar to the manner in which conductive paths are incorporated into silicon electronic chips or electrical circuit boards. First electrode/lead 202 and first wire 210, second electrode/lead 204 and second wire 212, third electrode/lead 206 and third wire 214, and fourth electrode/lead 208 and fourth wire 216 may be formed of a continuous line of conductive material. An electro-optical converter on the skin or close to electrodes/leads 202, 204, 206, 208 may allow use of optical fibers as wires instead of electrically conducting material.

First wire 210, second wire 212, third wire 214, and fourth wire 216 may comprise conducting fibers mounted within substrate 201, or mounted on substrate 201, such that there is no possibility that wires 210, 212, 214, 216 can contact the skin of the patient inadvertently. In an example embodiment, first wire 210, second wire 212, third wire 214, and fourth wire 216 are formed of carbon fiber or carbon rubber. In the example embodiment of FIG. 2, first wire 210, second wire 212, third wire 214, and fourth wire 216 are generally straight without curves or loops which may generate a current from the oscillating magnetic fields possibly generated by scanner 104. The carbon fiber or other conducting element forming first wire 210, second wire 212, third wire 214, and fourth wire 216 also can be wrapped in a tight spiral of the order of one millimeter (mm) to provide a sufficient inductance to prevent conduction of high frequency signals, e.g. greater than approximately 50 Hertz (Hz), that are substantially greater than the frequency of the heart beat which is typically one to two Hz. This also helps to avoid picking up induced currents which might cause a skin burn.

First wire 210, second wire 212, third wire 214, and fourth wire 216 may pass through substrate 201 without mounting to substrate 201. Wires 210, 212, 214, 216 may be formed of conductive strips or filaments insulated within substrate 201 and which lead to an edge of substrate 201 where they can be joined together to form cable 218. Wires 210, 212, 214, 216 may be formed of non-ferrous conducting material. Carbon rubber is an optimum material because it has good conductivity and is pliable, readily conforming to the variable shape of the chest or back. Wires 210, 212, 214, 216 may be formed of pliable material having elements that are configured to undergo plastic deformation to allow for adjustment to different sizes of patients and or different desired spacing between electrodes/leads 202, 204, 206, 208.

Cable 218 may be formed of carbon fibers which transport a signal from the patient through first electrode/lead 202, second electrode/lead 204, third electrode/lead 206, and fourth electrode/lead 208 to computing system 102. Cable 218 may be made of several conductive fibers and encased in a radio frequency (RF) shield to provide stiffness that minimizes curves or loops and electrical interference with the ECG gating signal. In an example embodiment, the signal is an analog signal.

In an example embodiment, cable 218 may further comprise a 50 inch plain copper cable with a five-prong connector, which eliminates the task of clipping alligator clips to ECG sensor device 200. Four six foot, two mm diameter, carbon fiber threads may be used to connect first electrode/lead 202, second electrode/lead 204, third electrode/lead 206, and fourth electrode/lead 208 to the copper wires in the plain cable portion of cable 218. Each individual carbon fiber thread may be insulated using six feet of 3/64 inch ID polyolefin tubing. All four insulated carbon fiber threads further may be insulated with six feet of RF-shielded polyolefin tubing. Each carbon fiber thread may be tied or looped onto the corresponding copper wire of the 50 inch plain cable. All bare wires may be shielded with polyolefin tubing. The 5-prong connector attached to a terminal end of the copper wire may plug into communication interface 109 of computing system 102.

In an alternative embodiment, the signal may be transmitted wirelessly to communication interface 109 of computing system 102, for example, using a Bluetooth protocol or other method of wireless data transmission at a frequency different from a frequency of scanner 104. When conducting signals wirelessly, the signal may be transmitted as an analog signal, or the signal may be first converted into a digital signal before transmission to computing system 102.

With reference to FIG. 3, an ECG sensor device 300 is shown in accordance with a second example embodiment. ECG sensor device 300 may include a substrate 301, a first electrode/lead 302, a second electrode/lead 304, a third electrode/lead 306, a fourth electrode/lead 308, a first wire 310, a second wire 312, a third wire 314, and a fourth wire 316. ECG sensor device 300 combines the ECG electrodes on a single device for rapid application to the patient. ECG sensor device 300 may include a fewer or a greater number of electrodes and corresponding wires. Substrate 301 is similar to substrate 201. For example, substrate 301 may comprise a sheet of non-conductive silicone or a viscoelastic foam block.

First electrode/lead 302, second electrode/lead 304, third electrode/lead 306, and fourth electrode/lead 308 are similar to first electrode/lead 202, second electrode/lead 204, third electrode/lead 206, and fourth electrode/lead 208. First electrode/lead 302, second electrode/lead 304, third electrode/lead 306, and fourth electrode/lead 308 are arranged to form a generally T-shaped array though oriented in a direction rotated approximately 90 degrees relative to the T-shaped array formed with reference to FIG. 2, though such an arrangement is not intended to be limiting. First electrode/lead 302, second electrode/lead 304, third electrode/lead 306, and fourth electrode/lead 308 may be positioned with a spacing of approximately two inches or more from one another.

First electrode/lead 302, second electrode/lead 304, third electrode/lead 306, and fourth electrode/lead 308 are connected or mount to first wire 310, second wire 312, third wire 314, and fourth wire 316, respectively, so that first wire 310, second wire 312, third wire 314, and fourth wire 316 conduct a signal detected by first electrode/lead 302, second electrode/lead 304, third electrode/lead 306, and fourth electrode/lead 308, respectively. First wire 310, second wire 312, third wire 314, and fourth wire 316 may be formed in a manner and of a material similar to that described with reference to first wire 210, second wire 212, third wire 214, and fourth wire 216. In the example embodiment of FIG. 3, however, first wire 210, second wire 212, third wire 214, and fourth wire 216 comprise tabs which protrude from substrate 301 to interface with alligator clips which can be connected to a cable that connects to computing system 102 through communication interface 109.

In the example embodiment of FIG. 3, first electrode/lead 302, second electrode/lead 304, third electrode/lead 306, and fourth electrode/lead 308 are generally circular spots having a diameter of approximately one inch and are formed of carbon-filled epoxy injected into substrate 301, which is comprised of a four inch by six inch sheet of uncured silicone gel. The conductive carbon-filled epoxy/gel may be created by mixing carbon black, graphite, and a binding agent. First wire 210, second wire 212, third wire 214, and fourth wire 216 comprise carbon fiber threads placed within substrate 301 such that one end is integrated into first electrode/lead 302, second electrode/lead 304, third electrode/lead 306, and fourth electrode/lead 308, respectively, and the remainder of the thread tunnels through the non-conductive silicone of substrate 301 out to a common region where the threads protrude approximately one inch from substrate 301 to interface with the alligator clips connected to the cable that connects to computing system 102 through communication interface 109. Alternatively, first wire 210, second wire 212, third wire 214, and fourth wire 216 may exit the edge of substrate 301 in a manner similar to that described with reference to FIG. 2.

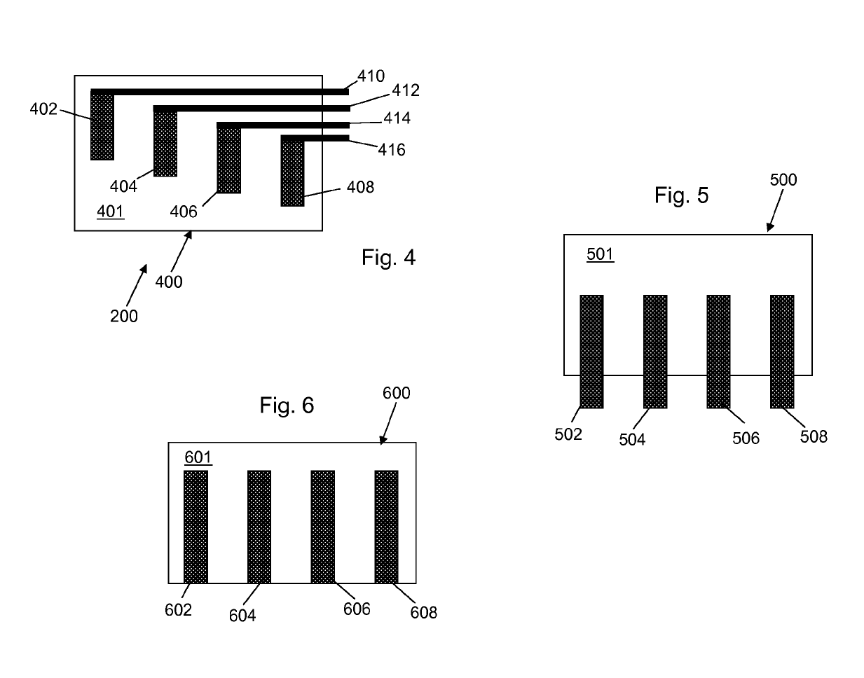

With reference to FIG. 4, an ECG sensor device 400 is shown in accordance with a third example embodiment. ECG sensor device 400 may include a substrate 401, a first electrode/lead 402, a second electrode/lead 404, a third electrode/lead 406, a fourth electrode/lead 408, a first wire 410, a second wire 412, a third wire 414, and a fourth wire 416. ECG sensor device 400 combines the ECG electrodes on a single device for rapid application to the patient. ECG sensor device 400 may include a fewer or a greater number of electrodes and corresponding wires. Substrate 401 is similar to substrate 201. For example, substrate 401 may comprise a sheet of non-conductive silicone or a viscoelastic foam block.

First electrode/lead 402, second electrode/lead 404, third electrode/lead 406, and fourth electrode/lead 408 are similar to first electrode/lead 202, second electrode/lead 204, third electrode/lead 206, and fourth electrode/lead 208. First electrode/lead 402, second electrode/lead 404, third electrode/lead 406, and fourth electrode/lead 408 are connected or mount to first wire 410, second wire 412, third wire 414, and fourth wire 416, respectively, so that first wire 410, second wire 412, third wire 414, and fourth wire 416 conduct a signal detected by first electrode/lead 402, second electrode/lead 404, third electrode/lead 406, and fourth electrode/lead 408, respectively. First wire 410, second wire 412, third wire 414, and fourth wire 416 may be formed in a manner and of a material similar to that described with reference to first wire 210, second wire 212, third wire 214, and fourth wire 216. In the example embodiment of FIG. 4, however, first wire 410, second wire 412, third wire 414, and fourth wire 416 comprise tabs which protrude from substrate 401 to interface with alligator clips which can be connected to the cable that connects to computing system 102 through communication interface 109.

In the example embodiment of FIG. 4, first electrode/lead 402 and first wire 410, second electrode/lead 404 and second wire 412, third electrode/lead 406 and third wire 414, and fourth electrode/lead 408 and fourth wire 416 together each form an L-shape. First electrode/lead 402, second electrode/lead 404, third electrode/lead 406, and fourth electrode/lead 408 each comprise a 2.5 inch by 0.5 inch conductive carbon rubber strip embedded in substrate 401 which comprises a four inch by six inch by 3/16 inch silicone sheet. First electrode/lead 402 is placed approximately 0.5 inch from a left edge of substrate 401, and fourth electrode/lead 408 is placed approximately 0.5 inch from a right edge of substrate 401 across the six inch length of substrate 401. First electrode/lead 402, second electrode/lead 404, third electrode/lead 406, and fourth electrode/lead 408 are spaced approximately one inch from one another. First wire 410, second wire 412, third wire 414, and fourth wire 416 comprise a carbon fiber thread or approximately ⅛ inch wide strip of carbon rubber used to conduct the electrical signal from each electrode/lead 402, 404, 406, 408 out to a common region where first wire 410, second wire 412, third wire 414, and fourth wire 416 protrude approximately one inch from substrate 401 to interface with alligator clips. In an example embodiment, first electrode/lead 402 and first wire 410, second electrode/lead 404 and second wire 412, third electrode/lead 406 and third wire 414, and fourth electrode/lead 408 and fourth wire 416 are set into a sheet of uncured silicone gel to integrate and secure the materials.

With reference to FIG. 5, an ECG sensor device 500 is shown in accordance with a fourth example embodiment. ECG sensor device 500 may include a substrate 501, a first electrode/lead 502, a second electrode/lead 504, a third electrode/lead 506, and a fourth electrode/lead 508. ECG sensor device 500 combines the ECG electrodes on a single device for rapid application to the patient. ECG sensor device 500 may include a fewer or a greater number of electrodes. Substrate 501 is similar to substrate 201. For example, substrate 501 may comprise a sheet of non-conductive silicone or a viscoelastic foam block.

First electrode/lead 502, second electrode/lead 504, third electrode/lead 406, and fourth electrode/lead 508 are similar to first electrode/lead 402, second electrode/lead 404, third electrode/lead 406, and fourth electrode/lead 408 except that first electrode/lead 502, second electrode/lead 504, third electrode/lead 406, and fourth electrode/lead 508 protrude approximately one inch from an edge of substrate 501 to interface with alligator clips which can be connected to the cable that connects to computing system 102 through communication interface 109.

With reference to FIG. 6, an ECG sensor device 600 is shown in accordance with a fourth example embodiment. ECG sensor device 600 may include a substrate 601, a first electrode/lead 602, a second electrode/lead 604, a third electrode/lead 606, and a fourth electrode/lead 608. ECG sensor device 600 combines the ECG electrodes on a single device for rapid application to the patient. ECG sensor device 600 may include a fewer or a greater number of electrodes. Substrate 601 is similar to substrate 201. For example, substrate 601 may comprise a sheet of non-conductive silicone or a viscoelastic foam block.

First electrode/lead 602, second electrode/lead 604, third electrode/lead 606, and fourth electrode/lead 608 are similar to first electrode/lead 402, second electrode/lead 404, third electrode/lead 406, and fourth electrode/lead 408 except first electrode/lead 602, second electrode/lead 604, third electrode/lead 606, and fourth electrode/lead 608 extend to an edge of substrate 501 without protruding therefrom though interfacing with alligator clips which can be connected to the cable that connects to computing system 102 through communication interface 109.

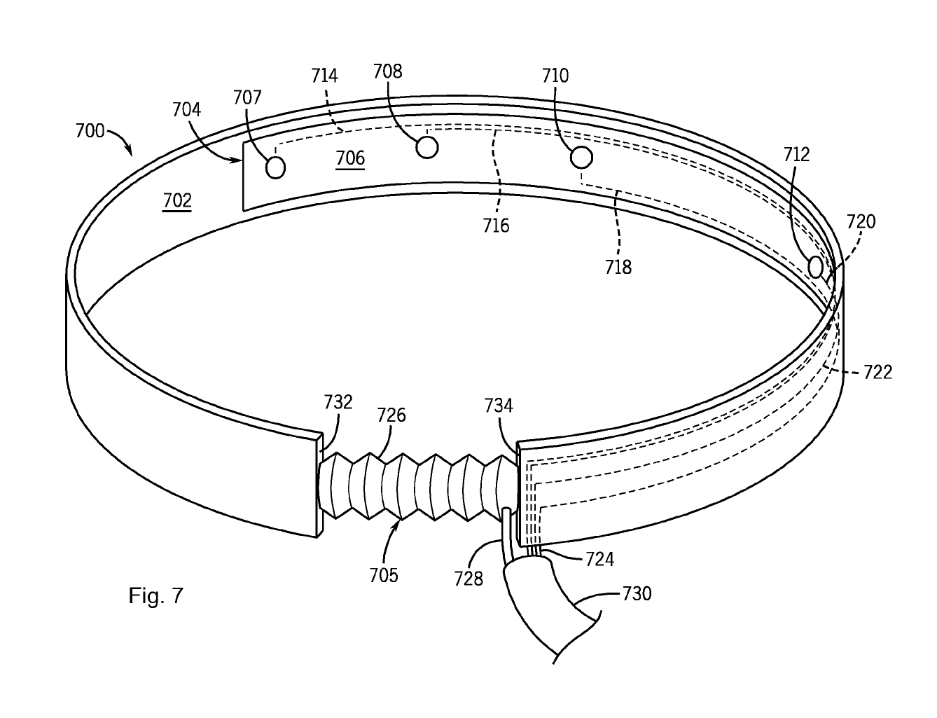

With reference to FIG. 7, an ECG/respiration sensor device 700 is shown in accordance with an example embodiment. ECG/respiration sensor device 700 may include a strap 702, an ECG sensor device 704, and a respiration sensor device 705. ECG sensor device 704 and respiration sensor device 705 mount to strap 702. Strap 702 is sized and shaped to wrap around the chest or abdomen of the patient. The fastening of strap 702 can be accomplished using hook-and-loop fasteners or other non-magnetic materials. Strap 702 may be non-elastic and depend on the elasticity of respiration sensor device 705 for a tight fit to the patient. ECG/respiration sensor device 700 is an example embodiment of ECG/respiration sensor device 106 which combines ECG sensor device 124 and respiration sensor device 126 in a single device for rapid application to the patient.

ECG sensor device 704 comprises at least one ECG electrode and may include one or more of ECG sensor device 200, ECG sensor device 300, ECG sensor device 400, ECG sensor device 500, and ECG sensor device 600. Additionally, without limitation and with reference to the example embodiment of FIG. 7, ECG sensor device 704 may include a substrate 706, a first electrode/lead 707, a second electrode/lead 708, a third electrode/lead 710, a fourth electrode/lead 712, a first wire 714, a second wire 716, a third wire 718, and a fourth wire 720. ECG sensor device 704 may include a fewer or a greater number of electrodes. Substrate 706 is similar to substrate 201. For example, substrate 706 may comprise a sheet of non-conductive silicone or a viscoelastic foam block.

In the example embodiment of FIG. 7, first electrode/lead 707, second electrode/lead 708, and third electrode/lead 710 are mounted on a surface of strap 702 such that, when strap 702 is wrapped and secured around the patient, first electrode/lead 707, second electrode/lead 708, and third electrode/lead 710 contact the back of the patient. Fourth electrode/lead 712 is mounted on a surface of strap 702 such that, when strap 702 is wrapped and secured around the patient, fourth electrode/lead 712 contacts the left side of the patient. First electrode/lead 707, second electrode/lead 708, third electrode/lead 710, and fourth electrode/lead 712 are generally distributed in a horizontal direction and sufficiently separated from one another to prevent short circuits between them. Typically, at least one cm spacing is sufficient to prevent short circuits. In the example embodiment of FIG. 7, first electrode/lead 707, second electrode/lead 708, third electrode/lead 710, and fourth electrode/lead 712 have a generally circular shape though other shapes may be used. Deformable material, such as viscoelastic foam, may be interposed between strap 702 and each electrode/lead 707, 708, 710, 712 to ensure contact with the skin. In an example embodiment, approximately one inch of viscoelastic foam may be used.

In the example embodiment of FIG. 7, first wire 714, a second wire 716, a third wire 718, and a fourth wire 720 have a high inductance to reduce the transmission of electrical signals greater than approximately 50 Hz and together form a cable 722 which extends to an edge of strap 702. Cable 722 is mounted to a second cable 724 which protrudes from strap 702.

Respiration sensor device 705 may comprise an accordion like tube 726 which expands and contracts with respiration of the patient to detect a respiratory motion of the patient. Accordion like tube 726 may include an elastic hollow portion that changes in size with patient respirations and may be mounted between a first end 732 of strap 702 and a second end 734 of strap 702. When strap 702 is wrapped and secured around the patient, accordion like tube 726 is positioned to the front of the patient generally across the chest or abdomen area. Respiration sensor device 705 may comprise a respiratory bellows, which may have a large cross-section, for example, greater than approximately one cm, to provide a greater airflow with each respiration making the respirations easier to detect.

In another example embodiment, respiration sensor device 705 may comprise two respiratory bellows integrated into strap 702. The two respiratory bellows may have a large cross-section, for example, greater than approximately one cm. One end of each bellows-strap can have hook-and-loop fasteners or other attachment mechanisms that enable attachment to the other bellows-strap or onto first end 732 of strap 702 or second end 734 of strap 702. As the patient breathes, only the respiratory bellows expand/contract because it is the only elastic component of strap 702. The length of the bellows-strap can be adjustable in order to accommodate the anatomical variability of patients.

Alternatively, respiration sensor device 705 may measure chest movement by sensing the stretching of at least one region of elastic within strap 702. Chest movement may also be detected by analysis of the variations in the electrical signals corresponding to chest wall motion such as the electrical activity of the intercostal or other muscles or variations in chest wall impedance.

In the example embodiment of FIG. 7, the pneumatic signal from respiration sensor device 705 is conducted through a third cable 728 which joins together with second cable 724 into a fourth cable 730 which is large enough to avoid getting caught in the various crevices of scanner 104 as the table on which the patient rests slides in and out of scanner 104. Combining the ECG gating and respiratory bellows into a single device allows fourth cable 730 carrying the first signal from respiration sensor device 705 and the second signal from ECG sensor device 704 to be combined into a single cable. This has the advantage of having just one cable to manage for transmitting a signal about respiration and about ECG from the patient to computing system 102 which interfaces with scanner 104. Fourth cable 730 may be relatively stiff with a slippery surface so that fourth cable 730 glides easily along a surface of scanner 104 as the patient is advanced in and out of scanner 104. The larger diameter of fourth cable 730 assists in keeping fourth cable 730 from getting caught in crevices between the sliding table and the fixed bed, which further minimizes the tension which second cable 724 places on ECG sensor device 704 and which third cable 728 places on respiration sensor device 705 thereby minimizing the risk of either device becoming disconnected. Because the crevices are typically about five mm or less in diameter, fourth cable 730 may have a diameter greater than approximately 5 mm, and preferably about one cm.

ECG/respiration sensor device 700 may include a marker to indicate a proper alignment of ECG/respiration sensor device 700 with the sternum of the patient so that the signals from electrodes/leads 707, 708, 710, 712 are optimally detected. ECG/respiration sensor device 700 can be removed and discarded or cleaned by swabbing with alcohol for reuse by the next patient in the same way that other reusable devices are cleaned for the next patient.

Sometimes, it is desirable to have electrodes/leads oriented vertically instead of horizontally as shown with reference to FIG. 7. In this case, a wide section of strap may allow the electrodes/leads to be oriented vertically. The distance between the electrodes/leads may be adapted for obese or pediatric patients or patients with large breasts. ECG sensor device 704 may include electrodes/leads on both the front and back and/or side of the chest of the patient to maximize the ECG signal. ECG sensor device 704 may include more electrodes/leads than are necessary with a mechanism to select which electrodes/leads are used after placement on the patient. In this way, if the ECG signal is not adequate, different electrodes/leads can be selected without having to move the patient out of the bore of scanner 104. When there are more electrodes/leads than are necessary, an algorithm included as part of data processing application 116 can evaluate the signal received from all of the electrodes/leads and automatically select the optimum combination for ECG gating. For example, the optimum combination of electrodes/leads may be the pairs which provide the maximum signal, maximum signal to noise ratio, the most stable signal, the signal least affected by RF and gradient switching electrical noise, etc. In another example embodiment, computing system 102 can display a signal corresponding to different combinations of electrodes/leads using display 118 and allow the operator to select the best combination, for example, using input interface 108 under control of data processing application 116 executed by processor 114. For example, the operator may select the combination which allows gating off the P wave instead of using the QRS wave to trigger the ECG signal earlier in the cardiac cycle.

In an example embodiment, strap 702 comprises a 37 inch by two inch non-elastic belt. The side of strap 702 facing away from the patient comprises a loop fastener. The side of strap 702 pressed against the patient includes four vertically oriented three inch by 0.5 inch conductive carbon electrodes integrated into a strip of silicone, or other non-conductive material, starting 1.5 inches from the right and spaced three inches from one another. The shape of strap 702 may be slightly conical to conform to the anatomy of the human abdomen. A 1.5 inch by 5.5 inch strip of hook fastener is mounted to first end 732 of strap 702 and used to fasten strap 702 to the patient.

In an example embodiment, the three inch by 0.5 inch conductive carbon electrodes are 2.5 mm thick though the thickness can vary from one mm to four mm depending on the mouth of the alligator clips that interfaces with the electrodes. A one inch tab of each carbon electrode protrudes from strap 702 and serves to interface with alligator clips to communicate with computing system 102 through communication interface 109. In an example embodiment, the electrodes have a hardness of approximately 60±5 as measured on a JISA hardness meter, a tensile strength of approximately 50 kilograms/cm², a tensile elongation of approximately 200%, a volume resistivity of approximately 5-10 ohms/cm, and a flammability of approximately UL94.

In an example embodiment, respiration sensor device 705 is embedded in first end 732 of strap 702, and respiration sensor device 705 has an approximately two cm² cross sectional area and is bent into a U-shape to double the cross-sectional area and to cover more chest/abdomen in the superior/inferior direction. Second end 734 of strap 702 may mount to respiration sensor device 705 by wrapping around the curve of the U and attaching onto itself. Within respiration sensor device 705, two rubber bands return the bellows back to their resting state after being stretched by inhalation. The bellows of respiration sensor device 705 may be composed of a corrugated (accordion-like) airtight shell made of a non-magnetic material that can expand and contract as the patient breathes. A ten foot long rubber tube may connect to the bottom of respiration sensor device 705 and attach to an air-pressure sensor via an appropriate press-fit air-tight connector.

In another example embodiment, one or two four inch by four inch foam/air filled elastic bladders are used in place of the bellows for respiratory monitoring. An elastic rubber mold can be used as well. The foam within the bladder may be a material with rapid shape recovery such as polyurethane, a viscoelastic material, etc. A valve that allows air into the bladder upon recovery may be used to further accelerate the bladder recovery time. When two bladders are used, a plastic “Y” connector may be used to connect both bladders to a ten foot rubber tube that attaches to the air-pressure sensor via an appropriate connector. One side of each bladder may be covered with a sheet of hook fasteners to attach to strap 702 and allow adjustment, when necessary. The bladders may be positioned such that they are atop the conductive electrodes/leads on the underside of strap 702 and press against the patient. Two bladders may be placed approximately two inches from first end 732 of strap 702 and approximately one inch from each other. As the chest expands, it compresses the bladder and forces air out into the sensor; as the chest contracts, the bladder draws air back into the bladder as it recovers. The bladder encasing the foam may be replaced by covering the foam with a flexible vinyl coating.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The word “example” is used herein to mean serving as an example, instance, or illustration. Any aspect or design described herein as “example” is not necessarily to be construed as preferred or advantageous over other aspects or designs. Further, for the purposes of this disclosure and unless otherwise specified, “a” or “an” means “one or more”. Still further, the use of “and” or “or” is intended to include “and/or” unless specifically indicated otherwise.

The foregoing description of example embodiments has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents

What is claimed is:

1. A device comprising:
a substrate; and
a plurality of electrodes mounted to the substrate and configured to sense an electrocardiogram signal of a patient when the plurality of electrodes are placed in contact with the patient.

2. The device of claim 1, wherein an electrode of the plurality of electrodes is formed of a non-ferrous conductive material.

3. The device of claim 2, wherein the non-ferrous conductive material is selected from the group consisting of silicon rubber, conductive epoxy, carbon black, and carbon rubber.

4. The device of claim 1, wherein an electrode of the plurality of electrodes has a circular shape having a diameter greater than or equal to two centimeters.

5. The device of claim 4, wherein the diameter greater than or equal to three centimeters and less than or equal to four centimeters.

6. The device of claim 1, wherein the plurality of electrodes are mounted to the substrate with a spacing of at least one centimeter between each electrode of the plurality of electrodes.

7. The device of claim 1, wherein an electrode of the plurality of electrodes has a rectangular shape having a length of at least 2.5 inches and a width of at least 0.5 inches.

8. The device of claim 1, wherein the plurality of electrodes extend from the substrate.

9. The device of claim 1, wherein the substrate is formed of a non-conductive material.

10. The device of claim 9, wherein the non-conductive material is selected from the group consisting of non-conductive silicone, silicon rubber, and plastic.

11. The device of claim 1, wherein the substrate comprises a biocompatible flexible material.

12. The device of claim 1, wherein the substrate comprises a deformable material.

13. The device of claim 12, wherein the deformable material is a viscoelastic foam.

14. The device of claim 13, wherein the deformable material is encased in a film.

15. The device of claim 1, further comprising a first wire and a second wire mounted within the substrate, wherein the plurality of electrodes comprises at least a first electrode and a second electrode, and further wherein the first wire connects to the first electrode and the second wire connects to the second electrode.

16. The device of claim 15, wherein the first wire and the second wire extend from the substrate.

17. The device of claim 15, wherein the first wire and the second wire connect to a common cable which extends from the substrate.

18. The device of claim 15, wherein the first wire comprises a non-ferrous conductive material.

19. The device of claim 15, further comprising an electro-optical converter connected between the first electrode and the first wire, wherein the first wire comprises an optical fiber.

20. The device of claim 1, further comprising a strap configured to wrap around a portion of the patient wherein the substrate mounts to the strap.

21. The device of claim 20, wherein the substrate is mounted to the strap in a position such that the plurality of electrodes are placed in contact with a back and a left side of the patient when the strap is positioned in contact with the patient.

22. The device of claim 20, further comprising a respiration sensor mounted to the strap.
